(19) United States
(12) Patent Application Publication
(10) Pub. No.: US 2014/0141504 A1
(43) Pub. Date: May 22, 2014

(54) GEL-FORMING DEVICE

(71) Applicant: SAMSUNG ELECTRO-MECHANICS CO., LTD., Suwon (KR)

(72) Inventors: Sang Hyun YI, Suwon (KR); Dong Woo LEE, Suwon (KR); Bo Sung KU, Suwon (KR)

(73) Assignee: SAMSUNG ELECTRO-MECHANICS CO., LTD., Suwon (KR)

(21) Appl. No.: 13/861,275

(22) Filed: Apr. 11, 2013

(30) Foreign Application Priority Data
Nov. 20, 2012 (KR) 10-2012-0131528

(51) Int. Cl. C12M 1/12

(52) U.S. Cl. CPC C12M 25/16
USPC 435/307.1

(57) ABSTRACT

There is provided a gel-forming device including a housing providing an inner space and having one opened side, a support plate disposed on one opened side of the housing and including at least one through-hole passing through the top and the bottom thereof, a substrate disposed on the top of the support plate and including at least one protrusion protruding from one surface thereof so as to have a biomaterial disposed thereon, and a spacing member provided on the bottom of the support plate so as to allow the support plate and the housing to be spaced apart from each other by a predetermined interval.

### CROSS-REFERENCE TO RELATED APPLICATIONS

This application claims the priority of Korean Patent Application No. 10-2012-0131528 filed on Nov. 20, 2012, in the Korean Intellectual Property Office, the disclosure of which is incorporated herein by reference.

### BACKGROUND OF THE INVENTION

1. Field of the Invention

The present invention relates to a gel-forming device, and more particularly, to a gel-forming device that may prevent a sol-state biomaterial from being evaporated during a gelation process.

2. Description of the Related Art

In recent years, with an increase in the necessity for research and development of biotechnology for rapidly diagnosing various human diseases, a bio chip or a cell chip required to examine a biomaterial has been steadily developed.

Since a bio chip or a cell chip is useful to examine a large quantity of biomaterials, a bio chip or a cell chip may even be used in a pharmaceutical company or a cosmetics company, as well as in a hospital.

A bio chip may be classified as a DNA chip, a protein chip, and a cell chip according to the type of a bio material fixed onto a substrate thereof, and may be constituted by a data chip where the biomaterial is cultured and a meta chip containing chemicals.

Methods in which a reaction of a cell to a predetermined drug are examined by coupling a data chip in which a biomaterial is cultured and the meta chip containing various chemical products or development reagents to each other, in order to examine a predetermined biomaterial have been used.

In this case, a sol-state biomaterial is attached to the data chip, and the sol-state biomaterial is altered to a gel-state biomaterial before coupling the data chip and the meta chip to each other.

Since the sol-state biomaterial is often evaporated during the gelation process, a study into a gel-forming device capable of preventing such a phenomenon and controlling the gelation process has been required.

### SUMMARY OF THE INVENTION

An aspect of the present invention provides a gel-forming device that may prevent a sol-state biomaterial from being contaminated and evaporated during a gelation process and easily control transition to a gel state.

According to an aspect of the present invention, there is provided a gel-forming device, including: a housing providing an inner space and having one opened side; a support plate disposed on one opened side of the housing and including at least one through-hole passing through the top and the bottom thereof; a substrate disposed on the top of the support plate and including at least one protrusion protruding from one surface thereof so as to have a biomaterial disposed thereon; and a spacing member provided on the bottom of the support plate so as to allow the support plate and the housing to be spaced apart from each other by a predetermined interval.

The inner space may have a liquid providing moisture to the biomaterial.

The protrusion may be provided on the substrate to correspond to the position of the through-hole.

The protrusion may be inserted into the through-hole to be positioned in the inner space.

The housing may include a bottom plate and a side wall part, and a step part may be provided on one end of the side wall part so that the support plate is seated on the step part.

According to another aspect of the present invention, there is provided a gel-forming device, including: a housing providing an inner space and having one opened side; a support plate disposed on one opened side of the housing and including at least one through-hole passing through the top and the bottom thereof; a substrate disposed on the top of the support plate and including at least one protrusion protruding from one surface thereof so as to have a biomaterial disposed thereon; and a cover part disposed on an upper side of the housing to be spaced apart from the housing by a predetermined interval.

The inner space may have a liquid therein providing moisture to the biomaterial.

The housing may include a bottom plate and a side wall part, and the side wall part is provided with a step part formed on one end thereof, the step part including the support plate seated thereon.

The cover part may include a top plate and an outer wall part, and the outer wall part may include a spacing member provided on the bottom thereof.

The support plate may include a communication hole formed therein enabling air in the inner space to be in communication with the outside.

According to another aspect of the present invention, there is provided a gel-forming device, including: a housing providing an inner space and having one opened side; a support plate disposed on one opened side of the housing and including at least one through-hole passing through the top and the bottom thereof; a substrate disposed on the top of the support plate and including at least one protrusion protruding from one surface so as to have a biomaterial disposed thereon; and a cover part coupled with the housing, wherein the cover part includes at least one ventilation hole formed therein to be in communication with the outside.

The inner space may have a liquid therein providing moisture to the biomaterial.

The support plate may include a communication hole enabling air in the inner space to be in communication with the outside.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is an exploded perspective view of a gel-forming device according to a first embodiment of the preset invention;

FIG. 2 is an exploded bottom perspective view of the gel-forming device according to the first embodiment of the preset invention;

FIG. 3 is an exploded perspective view of a gel-forming device according to a second embodiment of the preset invention;

FIG. 4 is an exploded bottom perspective view of the gel-forming device according to the second embodiment of the preset invention;

FIG. 5 is an exploded perspective view of a gel-forming device according to a third embodiment of the preset invention;

FIG. 6 is an exploded bottom perspective view of the gel-forming device according to the third embodiment of the preset invention;

FIG. 7 is an exploded perspective view of a gel-forming device according to a fourth embodiment of the preset invention; and

FIG. 8 is an exploded bottom perspective view of the gel-forming device according to the fourth embodiment of the preset invention.

### DETAILED DESCRIPTION OF THE INVENTION

Hereinafter, embodiments of the present invention will be described in detail with reference to the accompanying drawings. The invention may, however, be embodied in many different forms and should not be construed as being limited to the embodiments set forth herein.

Rather, these embodiments are provided so that this disclosure will be thorough and complete, and will fully convey the scope of the invention to those skilled in the art.

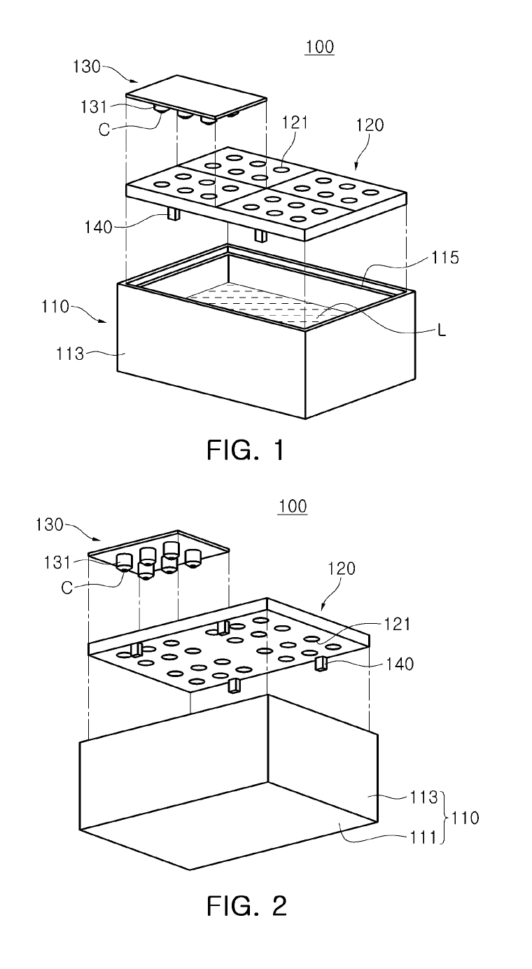

may FIG. 1 is an exploded perspective view of a gel-forming device according to a first embodiment of the preset invention. FIG. 2 is an exploded bottom perspective view of the gel-forming device according to the first embodiment of the preset invention.

Referring to FIGS. 1 and 2, a gel-forming device 100 according to the first embodiment of the present invention may include a housing 110, a support plate 120, a substrate 130, and a spacing member 140.

The housing 110 having a structure in which one side thereof is opened may include a bottom plate 111 and a side wall part 113 extending upwardly from an edge of the bottom plate 111.

That is, the housing 110 may have a structure in which the top thereof is opened and may provide an inner space surrounded by the bottom plate 111 and the side wall part 113.

A liquid L providing moisture to a biomaterial C to be described below may be filled in the inner space.

The support plate 120 may be disposed on one opened side of the housing 110.

When the support plate 120 is disposed on one opened side of the housing 110, a step part 115 may be provided on one end of the side wall part 113 provided in the housing 110 so that the support plate 120 is firmly seated on the housing 110.

That is, the support plate 120 is seated on the step part 115, and as a result, the support plate 120 may be firmly disposed on one opened side of the housing 110.

When the support plate 120 is disposed on one opened side of the housing 110, the spacing member 140 is provided between the support plate 120 and the housing 110, and in order to prevent the inner space of the housing 110 from being sealed, the support plate 120 may be spaced apart from the housing 110 by a predetermined interval.

In detail, the spacing member 130 may be provided on the bottom of the support plate 120 so as to contact the step part 115 provided on one end of the side wall part 113.

The support plate 120 may be spaced apart from the housing 110 by the height of the spacing member 140 through the spacing member 140.

Therefore, since a predetermined interval is formed between the support plate 120 and the housing 110, the housing 110 may not be sealed and air in the inner space of the housing may be in communication with the outside through the interval.

The support plate 120 may have a substantially thin plate shape as illustrated in FIG. 1.

In detail, the support plate 120 may have a rectangular parallelepiped shape having a predetermined length and a predetermined width and a through-hole 121 that passes through the top and the bottom of the support plate 120 may be provided on the support plate 120.

The amount of the through-holes 121 is not particularly limited in the present invention, and when the through-holes 121 are plural, the through-holes 121 may be formed on the support plate 120 at a predetermined interval.

The substrate 130 may be disposed on the top of the support plate 120 and a protrusion 131 provided on the substrate 130 may be inserted into the through-hole 121.

Therefore, the through-hole 121 and the protrusion 131 are provided on the support plate 120 and the substrate 130, respectively at positions corresponding to each other, and the amounts thereof may also correspond to each other.

The substrate 130 may have the substantially thin plate shape like the support plate 120 and may have the rectangular parallelepiped shape having the predetermined length and the predetermined width.

That is, the substrate 130 may have a shape corresponding to the support plate 120.

The substrate 130 may have the same length and width as those of the support plate 120, but is not limited thereto, that is, may also have a length and a width smaller than the support plate 120.

When the substrate 130 has the length and the width smaller than the support plate 120, a plurality of substrates are disposed on the support plate 120, and as a result, the length and width of all of the plurality of substrates may be the same as the length and width of the support plate 120, and the plurality of substrates may be disposed on the top of the support plate 120.

For reference, the substrate 130 used in the gel-forming device 100 according to the first embodiment of the present invention may be fabricated by using silicon, glass, metal, or polymer, but the present invention is not limited thereto.

The type of the polymer is not limited and may be, for example, polymethylmethacrylate (PMMA), polycarbonate (PC), polystyrene (PS), polypropylene, cyclic olefin copolymer, polynorbonene, styrene-butadien copolymer (SBC), or acrylonitrile butadiene styrene.

Further, a manufacturing method of the substrate 130 is not particularly limited. For example, the substrate 130 may be fabricated through a photoresist process, an etching process, an injection molding process, or the like.

Herein, at least one protrusion 131 protruding from one surface of the substrate 130 may be provided on the substrate 130.

The protrusion 131 is provided on a position corresponding to the through-hole 121 provided in the support plate 120, and the amount thereof may also be provided to correspond to the amount of the through-holes 121.

When the substrate 130 is disposed on the top of the support plate 120, the protrusion 131 is inserted into the through-hole 121, and as a result, one surface of the substrate 130 may be in close contact with the top of the support plate 120.

Here, the biomaterial C may be attached to the protrusion 131.

The biomaterial C may refer to various materials containing the biomaterial, without being limited to the biomaterial.

For example, a hexane sequence such as RNA or DNA, a peptide, a protein, a lipid, an organic or inorganic chemical molecule, a virus, procaryotic cell, or a cell organelle may be used instead of the biomaterial C, and the biomaterial C is mixed with a biopolymer such as alginate or matrigel to be attached to the protrusion 131.

Further, the biomaterial C is not limited to human cells and may be used in the sense of containing cells of various animals or plants.

Hereinafter, a process of gelling the sol-state biomaterial C by using the gel-forming device 100 according to the first embodiment of the present invention will be described.

From the following description, a configuration of the gel-forming device 100 according to the first embodiment of the present invention will also be described in more detail.

First, the sol-state biomaterial C is attached to the protrusion 131 provided on the substrate 131 and the liquid L providing the moisture is filled in the inner space of the housing 110.

Thereafter, when the support plate 120 is seated on the step part 115 provided on the side wall part 113 of the housing 110, the spacing member 140 provided on the bottom of the support plate 120 contacts the step part 115 and a space corresponding to the height of the spacing member 140 is formed between the support plate 120 and the housing 110.

The protrusion 131 provided on the substrate 130 faces the top of the support plate 120, and the substrate 130 is disposed on the top of the support plate 120 so that the protrusion 131 is inserted into the through-hole 121 provided on the support plate 120.

In this case, one surface of the substrate 130 and the top of the support plate 120 may in close contact with each other and the protrusion 131 to which the biomaterial C is attached may be positioned in the inner space provided by the housing 110.

Since the liquid L capable of providing the moisture to the biomaterial C is filled in the inner space, the biomaterial C positioned in the inner space may receive a predetermined amount of moisture from the liquid L.

When the biomaterial C is subjected to gelation while being left in the air, the biomaterial C may be evaporated, and when the biomaterial C is subjected to gelation within a sealed container, the biomaterial C is not in communication with outside air, and as a result, a gel state of the biomaterial C may not be controlled.

However, when the gel-forming device 100 according to the first embodiment of the present invention is used, the inner space provided by the housing 110 is not sealed and may be in communication with outside air through a predetermined interval formed between the support plate 120 and the housing 110, and since a predetermined amount of moisture may be provided to the biomaterial C by the liquid L filled in the inner space, the gelation process may be controlled.

That is, the gel-forming device 100 according to the first embodiment of the present invention has the liquid L that provides the moisture to the biomaterial C to prevent the biomaterial C from being evaporated, and the support plate 120 is disposed on one opened side of the housing 110 so as to be spaced apart from the housing 110 by a predetermined interval to prevent the biomaterial C from being contaminated.

Further, the sol-state biomaterial C may be easily controlled to be changed to the gel-state biomaterial C by controlling the amount of the liquid L filled in the inner space or a distance between the biomaterial C and the liquid L.

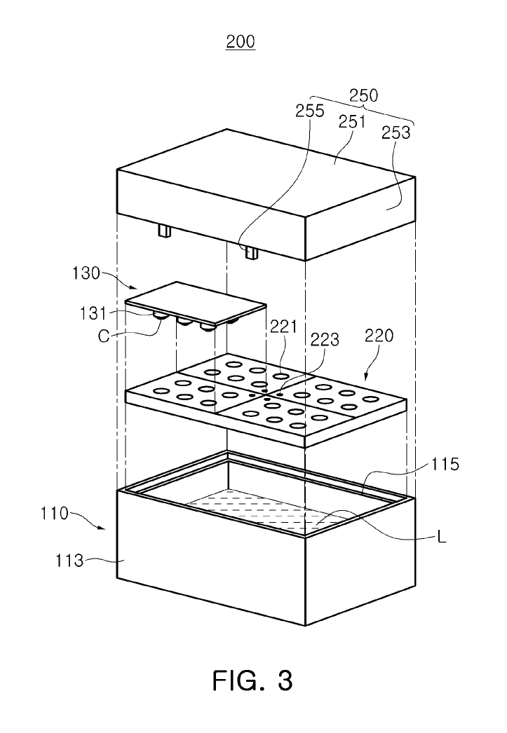

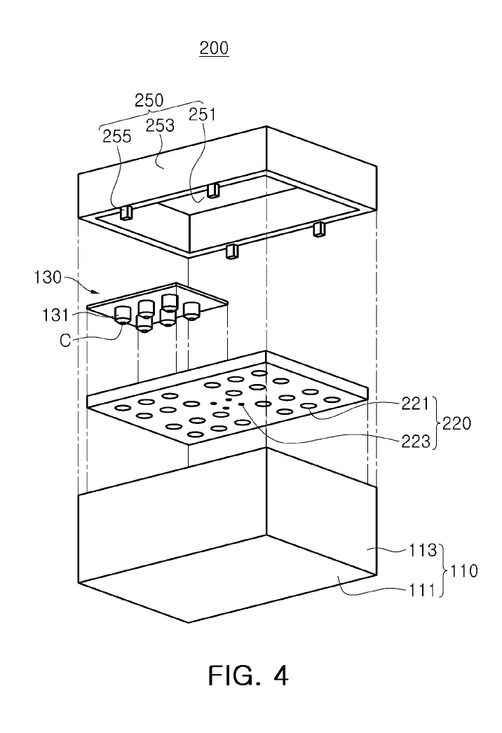

FIG. 3 is an exploded perspective view of a gel-forming device according to a second embodiment of the preset invention. FIG. 4 is an exploded bottom perspective view of the gel-forming device according to the second embodiment of the preset invention.

Referring to FIGS. 3 and 4, a gel-forming device 200 according to the second embodiment of the present invention may include a housing 110, a support plate 220, a substrate 130, and a cover part 250.

Since the gel-forming device 200 according to the second embodiment of the present invention includes the same components as the gel-forming device 100 according to the first embodiment except for the support plate 220 and the cover part 250, a description of the components other than the support plate 220 and the cover part 250 will be omitted.

The support plate 220 may be disposed on one opened side of the housing 110.

When the support plate 220 is disposed on one opened side of the housing 110, a step part 115 may be provided on one end of the side wall part 113 provided in the housing 110 so that the support plate 220 is firmly seated on the housing 110.

That is, the support plate 220 is seated on the step part 115, and as a result, the support plate 220 may be firmly disposed on one opened side of the housing 110.

When the support plate 220 is disposed on one opened side of the housing 110, the inner space of the housing 110 may be sealed, and as a result, at least one communication hole 223 which may be in communication with the outside may be provided in the support plate 220.

Since air in the inner space may be in communication with the outside of the housing 110 through the communication hole 223, the inner space is not sealed even in the case in which the support plate 220 is disposed on one opened side of the housing 110.

When the support plate 220 is seated on the step part 115, the top of the side wall part 113 provided in the housing 110 and the top of the support plate 220 may be positioned on the same plane.

The support plate 220 may have the substantially thin plate shape as illustrated in FIG. 3.

In detail, the support plate 220 may have the rectangular parallelepiped shape having a predetermined length and width and may include at least one communication hole 223 which may be in communication with the outside.

Further, a through-hole 221 that passes through the top and the bottom of the support plate 220 may be provided in the support plate 220.

The present invention is not particularly limited to the amount of the through-holes 221, and when the amount of the through-holes 221 are plural, the through-holes 221 may be formed in the support plate 220 with a predetermined interval therebetween.

The substrate 130 may be disposed on the top of the support plate 220 and a protrusion 131 provided on the substrate 130 may be inserted into the through-hole 221.

Therefore, the through-hole 221 and the protrusion 131 are respectively provided on the support plate 220 and the substrate 130, on positions corresponding to each other, and the amounts thereof may also correspond to each other.

The cover part 250 may be disposed on an upper side of the housing 110 to be spaced apart from the housing 110 by a predetermined interval.

In detail, the cover part 250 may include a top plate 251 and an outer wall part 253 extended downwardly from an edge of the top plate 251, and a protrusion 255 may be provided on the bottom of the outer wall part 253.

When the cover part 250 is disposed on the upper side of the housing 110, the protrusion 255 may contact the top of the side wall part 113 provided in the housing 110, and as a result, the cover part 250 and the housing 110 may be spaced apart from each other by a predetermined interval as high as the height of the protrusion 255.

Accordingly, since the air in the inner space of the housing 110 may be in communication with the outside through the communication hole 223 provided in the support plate 220, and a space formed between the cover part 250 and the housing 110, the inner space of the housing 110 may be not sealed.

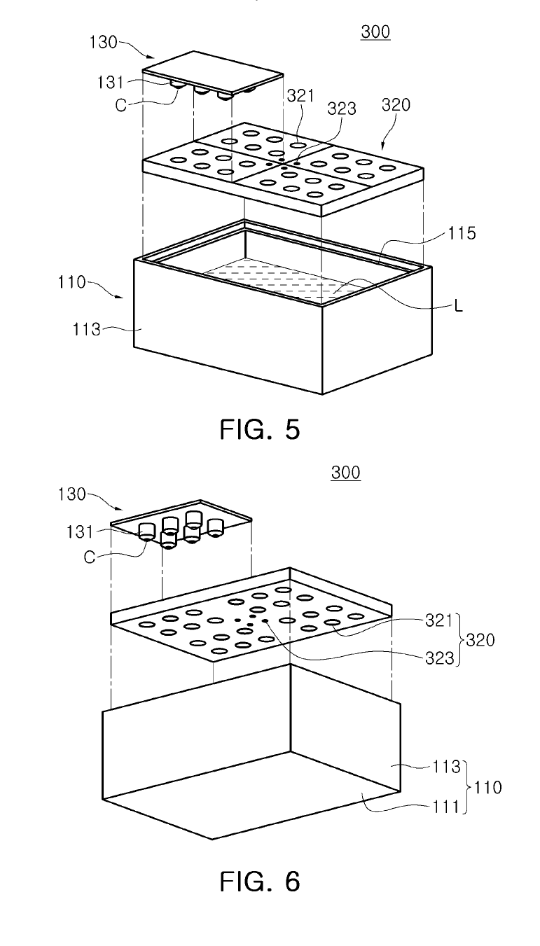

FIG. 5 is an exploded perspective view of a gel-forming device according to a third embodiment of the preset invention. FIG. 6 is an exploded bottom perspective view of the gel-forming device according to the third embodiment of the preset invention.

Referring to FIGS. 5 and 6, since a gel-forming device 300 according to the third embodiment of the present invention has the same components as the gel-forming device 100 according to the first embodiment, except for a support plate 320, a description of the components other than the support plate 320 will be omitted.

The support plate 320 may have the substantially thin plate shape as illustrated in FIG. 3 and the support plate 320 may have the rectangular parallelepiped shape having a predetermined length and width.

The support plate 320 may be disposed on one opened side of the housing 110, and the step part 115 may be provided on one end of the side wall part 113 provided in the housing 110 so that the support plate 320 is firmly seated on the housing 110.

That is, the support plate 320 is seated on the step part 115, and as a result, the support plate 320 may be firmly disposed on one opened side of the housing 110.

When the support plate 320 is disposed on one opened side of the housing 110, the inner space of the housing 110 may be sealed, and thus, at least one communication hole 323 which may be in communication with the outside may be provided in the support plate 320.

Since air in the inner space may be in communication with the outside of the housing 110 through the communication hole 323, the inner space is not sealed even in the case in which the support plate 320 is disposed on one opened side of the housing 110.

Further, a through-hole 321 that passes through the top and the bottom of the support plate 320 may be provided in the support plate 320.

The present invention is not particularly limited to the amount of the through-holes 321 provided according to the embodiment of the present invention, and when the through-holes 321 are plural, the through-holes 321 may be formed in the support plate 320 to be spaced apart from each other by a predetermined interval.

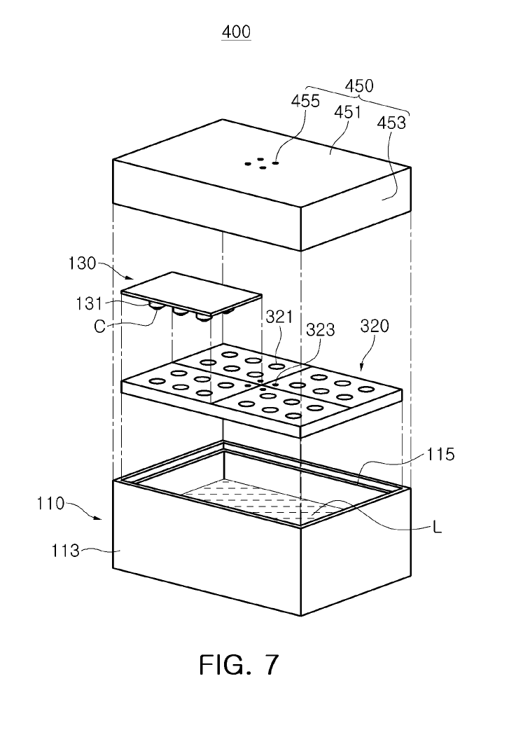

FIG. 7 is an exploded perspective view of a gel-forming device according to a fourth embodiment of the preset invention.

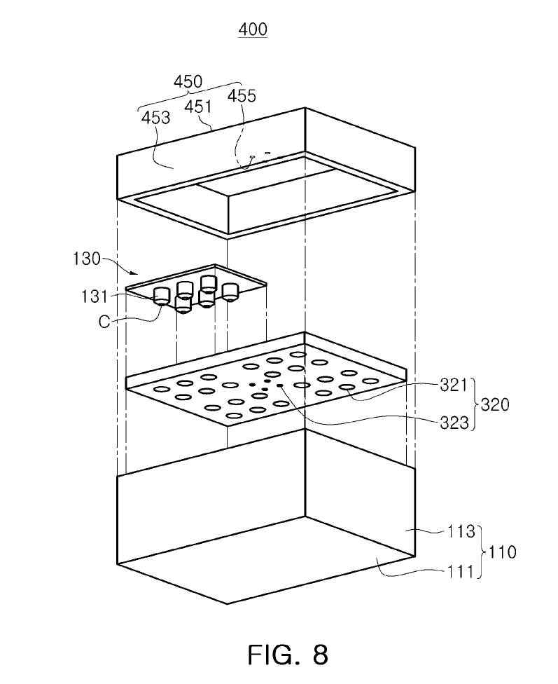

FIG. 8 is an exploded bottom perspective view of the gel-forming device according to the fourth embodiment of the preset invention.

Referring to FIGS. 7 and 8, since a gel-forming device 400 according to the fourth embodiment of the present invention has the same components as the gel-forming device 300 according to the third embodiment, except for a cover part 450, a description of the components other than the cover part 450 will be omitted.

The cover part 450 may include a top plate 451 and an outer wall part 453 extended downwardly from an edge of the top plate 451, and the cover part 450 may be coupled to the housing 110.

The outer wall part 453 of the cover part 450 and the side wall part 113 of the housing 110 are disposed to contact each other, such that the cover part 450 may be coupled to the housing 110.

In this case, at least one ventilation hole 455 which may be in communication with the outside may be provided in the cover part 450, and since the air in the inner space provided by the housing 110 may be in communication with the outside through the communication hole 323 provided in the support plate 320 and the ventilation hole 455 provided in the cover part 450, the inner space of the housing 110 may not be sealed.

Through the embodiments, the gel-forming device according to the embodiments of the present invention has the liquid providing the moisture to the biomaterial to prevent the biomaterial from being evaporated and includes the support plate or the cover part disposed on one opened side of the housing to prevent the biomaterial from being contaminated.

Further, the sol-state biomaterial may be easily controlled to be changed to the gel-state biomaterial by controlling the amount of the liquid L in the gel-forming device or the distance between the biomaterial and the liquid.

As set forth above, with the gel-forming device according to the embodiment of the present invention, the sol-state biomaterial may be prevented from being contaminated and evaporated during the gelation process and the transition to the gel state may be easily controlled.

While the present invention has been shown and described in connection with the embodiments, it will be apparent to those skilled in the art that modifications and variations can be made without departing from the spirit and scope of the invention as defined by the appended claims.

What is claimed is:

1. A gel-forming device, comprising:
a housing providing an inner space and having one opened side;
a support plate disposed on one opened side of the housing and including at least one through-hole passing through the top and the bottom thereof;
a substrate disposed on the top of the support plate and including at least one protrusion protruding from one surface thereof so as to have a biomaterial disposed thereon; and
a spacing member provided on the bottom of the support plate so as to allow the support plate and the housing to be spaced apart from each other by a predetermined interval.

2. The gel-forming device of claim 1, wherein the inner space has a liquid providing moisture to the biomaterial.

3. The gel-forming device of claim 1, wherein the protrusion is provided on the substrate to correspond to the position of the through-hole.

4. The gel-forming device of claim 1, wherein the protrusion is inserted into the through-hole to be positioned in the inner space.

5. The gel-forming device of claim 1, wherein the housing includes a bottom plate and a side wall part, and a step part is provided on one end of the side wall part so that the support plate is seated on the step part.

6. A gel-forming device, comprising:
a housing providing an inner space and having one opened side;
a support plate disposed on one opened side of the housing and including at least one through-hole passing through the top and the bottom thereof;
a substrate disposed on the top of the support plate and including at least one protrusion protruding from one surface thereof so as to have a biomaterial disposed thereon; and
a cover part disposed on an upper side of the housing to be spaced apart from the housing by a predetermined interval.

7. The gel-forming device of claim 6, wherein the inner space has a liquid therein providing moisture to the biomaterial.

8. The gel-forming device of claim 6, wherein the housing includes a bottom plate and a side wall part, and the side wall part is provided with a step part formed on one end thereof, the step part including the support plate seated thereon.

9. The gel-forming device of claim 6, wherein the cover part includes a top plate and an outer wall part, and the outer wall part includes a spacing member provided on the bottom thereof.

10. The gel-forming device of claim 6, wherein the support plate includes a communication hole formed therein enabling air in the inner space to be in communication with the outside.

11. A gel-forming device, comprising:
a housing providing an inner space and having one opened side;
a support plate disposed on one opened side of the housing and including at least one through-hole passing through the top and the bottom thereof;
a substrate disposed on the top of the support plate and including at least one protrusion protruding from one surface so as to have a biomaterial disposed thereon; and
a cover part coupled with the housing,
wherein the cover part includes at least one ventilation hole formed therein to be in communication with the outside.

12. The gel-forming device of claim 11, wherein the inner space has a liquid therein providing moisture to the biomaterial.

13. The gel-forming device of claim 11, wherein the support plate includes a communication hole enabling air in the inner space to be in communication with the outside.
